(19) United States
(12) Patent Application Publication
(10) Pub. No.: US 2024/0358720 A1
(43) Pub. Date: Oct. 31, 2024

(54) Dosing Regimen for a TEAD inhibitor

(71) Applicant: Novartis AG, Basel (CH)

(72) Inventor: Emilie CHAPEAU, Huningue (FR)

(21) Appl. No.: 18/687,599

(22) PCT Filed: Aug. 30, 2022
(86) PCT No.: PCT/IB2022/058130

§ 371 Date: Feb. 28, 2024

Related U.S. Application Data
(60) Provisional application No. 63/239,506, filed on Sep. 1, 2021.

(51) Int. Cl. A61K 31/635; A61P 13/12; A61P 35/00

(52) U.S. Cl. CPC A61K 31/635; A61P 13/12; A61P 35/00

(57) ABSTRACT

The invention relates to a TEAD inhibitor or a pharmaceutically acceptable salt thereof for use in the treatment of cancer, wherein the TEAD inhibitor is administered on each of the first 3 days of a 7 day treatment cycle, and wherein the treatment comprises at least two treatment cycles.

### FIELD OF THE DISCLOSURE

The present invention relates to a TEAD inhibitor or a pharmaceutically acceptable salt thereof for use in the treatment of cancer, and with specific dosing regimens, e.g., the TEAD inhibitor is administered on each of the first 3 days of a 7 day treatment cycle, and wherein the treatment comprises at least two treatment cycles.

### BACKGROUND

Normal tissue growth, as well as tissue repair and remodeling, require specific control and regulated balance of transcriptional activity. Transcriptional output is coordinated through a number of key signaling modules, one of which is the Hippo pathway. Genetic studies in Drosophila and mammals have defined a conserved core signaling cassette, composed of Mst1/2 and Lats1/2 kinases which inhibit the transcriptional co-activators YAP and TAZ (official gene name: WWTR1).

An activated Hippo pathway translates to YAP and TAZ being phosphorylated and sequestered/degraded in the cytoplasm. Upon inactivation of the Hippo pathway, YAP and TAZ translocate to the nucleus and associate with transcription factors, namely members of the TEAD family (TEAD1-4). The YAP/TAZ-TEAD complexes in turn promote transcription of downstream genes involved in cellular proliferation, death and differentiation. While YAP and TAZ can also interact with a number of other factors, TEADs are commonly accepted to be the key mediators of the growth-promoting and tumorigenic potential of YAP and TAZ (pathway reviewed in Yu et al., 2015; Holden and Cunningham, 2018).

Accordingly, a hyperactivation of YAP and/or TAZ (and subsequent hyperactivity of the YAP/TAZ-TEAD transcriptional complex) is commonly observed in several human cancers. This is evidenced by the levels and nuclear localization of YAP/TAZ being elevated in many tumors, including breast, lung (e.g., non-small cell; NSCLC), ovarian, colorectal, pancreas, prostate, gastric, esophagus, liver and bone (sarcoma) (Steinhardt et al., 2008; Harvey et al., 2013; Moroishi et al., 2015; extensively reviewed in Zanconato et al., 2016 and references therein). While genetic alterations of the core Hippo pathway components have thus far been detected with limited frequency in primary samples, the most prominent cancer malignancy associated with inactivating mutations in NF2 or Lats1/2 and associated YAP/TEAD hyperactivity is malignant pleural mesothelioma (MPM) (reviewed in Sekido, 2018). Similarly, a number of human tumors are characterized by amplification of YAP at the 11922.1 locus (e.g., hepatocellular carcinomas, medulloblastomas, esophageal squamous cell carcinomas), TAZ (WWTR1) at the 3q25.1 locus (e.g., rhabdomyosarcomas, triple negative breast cancer) or gene fusions involving YAP or TAZ (epithelioid hemangioendotheliomas, ependymal tumors) (reviewed in Yu et al., 2015 and references therein). As is the case for MPM, such tumors are also anticipated to depend on their elevated YAP/TAZ-TEAD activity.

Disruption of the YAP/TAZ-TEAD PPI as the most distal effector node of the Hippo pathway is anticipated to abolish the oncogenic potential of this complex. The compounds of this invention are designed and optimized to bind to TEADs and selectively disrupt their interaction with YAP and TAZ, which is believed to result in drugs useful in the treatment of above-mentioned cancers. In particular, such cancers may be characterized by (but not restricted to) some of the described aberrations.

Notably, tumor cells with activated YAP/TAZ-TEAD display resistance to chemotherapeutic drugs, possibly related to YAP/TAZ conferring cancer stem cell-like characteristics. Moreover, YAP/TAZ-TEAD activation also confers resistance to molecularly targeted therapies, such as BRAF, MEK or EGFR inhibitors, as reported from the outcome of various genetic and pharmacological screens (Kapoor et al., 2014; Shao et al., 2014; Lin et al., 2015). This in turn suggests that inhibiting YAP/TAZ-TEAD activity—either in parallel or sequentially to other cancer treatments—may provide a beneficial therapeutic impact by reducing growth of tumors resistant to other treatments. The inhibition of YAP/TAZ-TEAD activity upon PPI disruption with above mentioned LMW compounds may also blunt the tumor's escape from immune surveillance. This is, for instance, evidenced by reported data on YAP promoting the expression of chemokine CXCL5 which results in the recruitment of myeloid cells that suppress T-cells (Wang et al., 2016). YAP in Tregs (regulatory T-cells) has also been demonstrated to support FOXP3 expression via activin signaling and Treg function. Accordingly, YAP deficiency results in dysfunctional Tregs which are no longer able to suppress antitumor immunity. Selective inhibition of YAP/TEAD activity may therefore contribute to bolster antitumor immunity by preventing Treg function (Ni et al., 2018). Recent literature also suggests that YAP upregulates PD-L1 expression and by this mechanism directly mediates evasion of cytotoxic T-cell immune responses, for instance in BRAF inhibitor-resistant melanoma cells (Kim et al., 2018). For treatment purposes, above-mentioned YAP/TAZ-TEAD PPI compounds may be used in combination with cancer immunotherapy drugs, such as immune checkpoint inhibitors (e.g., anti-PD-1 antibodies).

The TEAD inhibitor 4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide (Compound A), and methods of preparing said inhibitor are described in International Patent Application PCT/IB2021/052136, which is incorporated by reference.

Kidney toxicity, in particular tubular degeneration, is thought to be a safety/toxicity risk associated with some TEAD inhibitors, especially in the case of long term treatment. Thus, there remains a need in the art for dosing regimens of TEAD inhibitors that result in an improved safety profile.

### See for Example

Yu, F-X., Zhao, B. and Guan, K.-L. (2015). Hippo pathway in organ size control, tissue homeostasis, and cancer. Cell, 163, 811-828.
Holden, J. K. and Cunningham, C. N. (2018). Targeting the Hippo pathway and cancer through the TEAD family of transcription factors. Cancers (Basel), 10, E81.
Steinhardt, A. A., Gayyed, M. F., Klein, A. P., Dong, J., Maitra, A., Pan, D., Montgomery, E. A., Anders, R. A. (2008). Expression of Yes-associated protein in common solid tumors. Hum. Pathol., 39, 1582-1589.
Harvey, K. F., Zhang, X., and Thomas, D. M. (2013). The Hippo pathway and human cancer. Nat. Rev. Cancer, 13, 246-257.
Moroishi, T., Hansen, C. G., and Guan, K.-L. (2015). Nat. Rev. Cancer, 15, 73-79.
Zanconato, F., Cordenonsi, M., and Piccolo, S. (2016). YAP/TAZ at the roots of cancer. Cancer Cell, 29, 783-803.
Sekido, Y. (2018). Cancers (Basel), 10, E90.
Kapoor, A., Yao, W., Ying, H., Hua, S., Liewen, A., Wang, Q., Zhong, Y., Wu, C. J., Sadanandam, A., Hu, B. et al. (2014). Yap1 activation enables bypass of oncogenic Kras addiction in pancreatic cancer. Cell, 158, 185-197.
Shao, D. D., Xue, W., Krall, E. B., Bhutkar, A., Piccioni, F., Wang, X., Schinzel, A. C., Sood, S., Rosenbluh, J., Kim, J. W., et al. (2014). KRAS and YAP1 converge to regulate EMT and tumor survival. Cell, 158, 171-184.
Lin, L., Sabnis, A. J., Chan, E., Olivas, V., Cade, L., Pazarentzos, E., Asthana, S., Neel, D., Yan, J. J., Lu, X. et al. (2015). The Hippo effector YAP promotes resistance to RAF- and MEK-targeted cancer therapies. Nat. Genet., 47, 250-256.
Wang, G., Lu, X., Dey, P., Deng, P., Wu, C. C., Jiang, S., Fang, Z., Zhao, K., Konaprathi, R., Hua, S., et al. (2016). Cancer Discov., 6, 80-95.
Ni, X., Tao, J., Barbi, J., Chen, Q., Park B. V., Li, Z., Zhang, N., Lebid, A., Ramaswamy, A., Wei, P., et al. (2018). YAP is essential for Treg-mediated suppression of antitumor immunity. Cancer Discov., 8, 1026-1043.
Kim, M. H., Kim, C. G., Kim, S. K., Shin, S. J., Choe, E. A., Park, S. H., Shin, E. C., and Kim, J. (2018). Cancer Immunol Res., 6, 255-266.

### SUMMARY

One of the objectives in the development of TEAD inhibitors is to find a dosing regimen which ensures efficacy but at the same time is associated with a reduced amount of adverse side effects (e.g. kidney toxicity).

It has been surprisingly found that the dosing schedule of the present invention is associated with reduced kidney toxicity, compared to a continuous daily dosing schedule.

Specifically, the present invention provides the following aspects, advantageous features and specific embodiments, alone or in combination, as listed in the following numbered embodiments.

Embodiment 1. A TEAD inhibitor or a pharmaceutically acceptable salt thereof for use in the treatment of cancer, wherein the TEAD inhibitor or pharmaceutically acceptable salt thereof is administered on each of the first 3 days of a 7 day treatment cycle, and wherein the treatment comprises at least two treatment cycles.

Embodiment 2. A method of treating cancer in a subject in need thereof, wherein the method comprises administering to the subject a therapeutically effective amount of a TEAD inhibitor, or a pharmaceutically acceptable salt thereof on each of the first 3 days of a 7 day treatment cycle, and wherein the treatment comprises at least two treatment cycles.

Embodiment 3. A method of reducing albuminuria and/or kidney toxicity in a subject undergoing treatment with a TEAD inhibitor or a pharmaceutically acceptable salt thereof, wherein the method comprises administering to the subject a therapeutically effective amount of the TEAD inhibitor, or a pharmaceutically acceptable salt thereof, on each of the first 3 days of a 7 day treatment cycle, and wherein the treatment comprises at least two treatment cycles.

Embodiment 4. The TEAD inhibitor or pharmaceutically acceptable salt thereof for use in the treatment of cancer according to Embodiment 1, or the method according to Embodiment 2 or Embodiment 3, wherein the TEAD inhibitor is a YAP/TAZ-TEAD protein/protein interaction inhibitor or a pharmaceutically acceptable salt thereof.

Embodiment 5. The TEAD inhibitor or pharmaceutically acceptable salt thereof for use in the treatment of cancer according to Embodiment 1 or Embodiment 4, or the method according to any one of Embodiments 2 to 4, wherein the TEAD inhibitor or salt thereof is 4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide or a pharmaceutically acceptable salt thereof.

Embodiment 6. The TEAD inhibitor or pharmaceutically acceptable salt thereof for use in the treatment of cancer according to any one of Embodiments 1, 4 and 5, or the method according to any one of Embodiments 2 to 5, wherein the daily dose on each administration day is from 15 mg to 100 mg.

Embodiment 6a. The TEAD inhibitor or pharmaceutically acceptable salt thereof for use in the treatment of cancer according to any one of Embodiments 1, 4 and 5, or the method according to any one of Embodiments 2 to 5, wherein the daily dose on each administration day is from 15 mg to 500 mg, for example from 60 mg to 300 mg, for example from 60 mg to 240 mg.

Embodiment 7. The TEAD inhibitor or pharmaceutically acceptable salt thereof for use in the treatment of cancer according to Embodiment 6, or the method according to Embodiment 6, wherein the daily dose on each administration day is 15, 30, 45, 60, 75 mg, 90 mg or 100 mg.

Embodiment 8. The TEAD inhibitor or pharmaceutically acceptable salt thereof for use in the treatment of cancer according to any one of Embodiments 1 and 4 to 7, or the method according to any one of Embodiments 2 to 7, wherein the cancer is a TEAD dependent cancer (e.g. wherein the cancer has a Hippo pathway dysregulation) or is a solid tumor with NF2/LATS1/LATS2 mutations.

Embodiment 9. The TEAD inhibitor or pharmaceutically acceptable salt thereof for use in the treatment of cancer according to any one of Embodiments 1 and 4 to 8, or the method according to any one of Embodiments 2 to 8, wherein the cancer is selected from breast cancer, lung cancer, ovarian cancer, kidney cancer, uterine cancer, colorectal cancer, mesothelioma, e.g. malignant pleural mesothelioma, pancreatic cancer, prostate cancer, gastric cancer, esophageal cancer, liver cancer, medulloblastoma, head and neck cancer, sarcoma, epithelioid hemangioendothelioma, ependymal tumor and bone cancer, e.g. wherein the cancer is mesothelioma, e.g. wherein the cancer is malignant pleural mesothelioma.

The dosing regimens of the present invention provide for a reduced kidney toxicity, as illustrated in the Drawings and Examples.

### BRIEF DESCRIPTION OF THE DRAWINGS

In the following, the present invention is described in detail with reference to the accompanying figures in which:

FIG. 1 shows (A) antitumor activity, (B) survival and (C-E) urine kidney injury marker evaluation of Compound A in a MSTO-211H s.c. xenograft rat model, using a weekly dose of 420 mg/kg p.o. following one of four different Compound A dosing schedules or vehicle control.

FIG. 2 shows the comparative antitumor efficacy and tolerability of Compound A with daily or intermittent schedules in nude rats bearing (A) MSTO-211H and (B) NCI-H226 mesothelioma tumors. Values are mean±SEM; sample size: n=5. *p<0.05, significant inhibition compared to vehicle control group (One-way ANOVA, with Dunnett's multiple comparison tests on tolerability data). (C) Urine kidney injury marker evaluation of Compound A, the results from the two experiments are combined, from the MSTO-211H and NCI-H226 s.c. xenograft rat models, Values are mean±SEM; sample size: n=4-6. *p<0.05, **p<0.01, ***p<0.001 significant inhibition compared to vehicle control group (One-way ANOVA, with Dunnett's multiple comparison tests).

### DETAILED DESCRIPTION

As explained above, one of the objectives in the development of TEAD inhibitors is to find a dosing regimen which ensures efficacy but at the same time is associated with a reduced amount of adverse side effects (e.g. kidney toxicity).

The invention therefore provides a TEAD inhibitor or a pharmaceutically acceptable salt thereof for use in the treatment of cancer, wherein the TEAD inhibitor or pharmaceutically acceptable salt thereof is administered on each of the first 3 days of a 7 day treatment cycle, and wherein the treatment is composed of at least two treatment cycles. As explained in the Examples and Drawings, this has been found to be equivalent to QD dosing in terms of efficacy, but at the same time result in improved survival and reduced kidney toxicity, thus leading to a larger therapeutic window.

It should be understood by “administered on each of the first 3 days of a 7 day treatment cycle”, it is meant that the TEAD inhibitor or pharmaceutically acceptable salt thereof is administered on each of the first 3 days of the 7 day treatment cycle, and not on the subsequent 4 days of the 7 day treatment cycle.

Preferably, the at least two treatment cycles are consecutive, that is to say the second treatment cycle follows immediately on from the first treatment cycle. For example, the invention therefore includes the following:
Days 1-3: TEAD inhibitor administered on each day;
Days 4-7: TEAD inhibitor not administered;
Days 8-10: TEAD inhibitor administered on each day; and
Days 11-14: TEAD inhibitor not administered.

In this example, days 1-3 and 8-10 are administration days. An “administration day” thus refers to any day where the TEAD inhibitor is administered to the patient.

When present, the third (fourth etc.) treatment cycles preferably immediately follow on from the previous treatment cycle. Thus, in an embodiment where there are three treatment cycles, the invention includes the following:
Days 1-3: TEAD inhibitor administered on each day;
Days 4-7: TEAD inhibitor not administered;
Days 8-10: TEAD inhibitor administered on each day;
Days 11-14: TEAD inhibitor not administered;
Days 15-17: TEAD inhibitor administered on each day; and
Days 18-21: TEAD inhibitor not administered.

In an embodiment, there are three or more treatment cycles, e.g. four or more treatment cycles, e.g. five or more treatment cycles, e.g. six or more treatment cycles, e.g. eight or more treatment cycles, e.g. ten or more treatment cycles.

The TEAD inhibitor may be present as a free molecule, or as a pharmaceutically acceptable salt thereof. Preferably, the TEAD inhibitor is present in free form (i.e. not a salt), and is optionally solvated.

The term “TEAD inhibitor” denotes any compound inhibiting the TEAD protein with an IC₅₀ of less than 10 μM, preferably less than 1 μM, more preferably less than 0.1 μM, even more preferably less than 0.01 μM measured by a Time Resolved Fluorescence Energy Transfer (TR-FRET) Assay.

As used herein, the term “YAP/TAZ-TEAD PPII” or “YAP/TAZ-TEAD Protein-Protein Interaction Inhibitor” or “YAP/TAZ-TEAD PPI Inhibitor” refers to a compound which is capable of inhibiting the interaction between i) TEAD and ii) YAP and/or TAZ, for example by binding to TEAD and thus selectively disrupting TEAD's interaction with YAP and/or TAZ. In an embodiment, the IC₅₀ is less than 10 μM, preferably less than 1 μM, more preferably less than 0.1 μM, even more preferably less than 0.01 μM measured by a Time Resolved Fluorescence Energy Transfer (TR-FRET) Assay.

As used herein, the term “daily dose” refers to the total dosage amount administered to an individual in a single 24-hour day.

When referring to a dose amount of the TEAD inhibitor herein, e.g. in mg (milligrams), it is meant as the (equivalent) amount of the TEAD inhibitor in free form (i.e. excluding, for instance, the salt or co-crystal partner as well as any solvent present).

As used herein, the terms “salt” or “salts” refers to an acid addition or base addition salt of a conjugate of the present invention. “Salts” include in particular “pharmaceutical acceptable salts”. The term “pharmaceutically acceptable salts” refers to salts that retain the biological effectiveness and properties of the conjugate of this invention and, which typically are not biologically or otherwise undesirable. In many cases, the conjugates of the present invention are capable of forming acid and/or base salts by virtue of the presence of amino and/or carboxyl groups or groups similar thereto. When both a basic group and an acid group are present in the same molecule, the conjugates of the present invention may also form internal salts, e.g., zwitterionic molecules. Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, the present invention provides conjugates of the present invention in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

Preferably, the drug administration is done by oral delivery, i.e. oral administration, per oral (p.o.).

Preferably, the drug is provided in the form of an oral dosage form, more preferably in the form of a solid oral dosage form, e.g. a capsule or a tablet.

Preferably the drug is taken with a glass of water and without chewing the capsules or tablet.

If the patient is assigned to a dose level where multiple capsules/tablets are to be taken, the capsules/tablets should be taken consecutively, within as short a time interval as possible, e.g. within 5 minutes.

Preferably, the drug is administered at approximately the same time each administration day. Preferably, the drug is administered once daily on each administration day. More preferably, the drug is administered in the morning.

Preferably, the drug is administered in the fasted state, i.e. at least 1 hour before or 2 hours after a meal.

The term “drug” as used herein refers to the TEAD inhibitor, or pharmaceutically acceptable salt thereof.

The TEAD inhibitor can be delivered to the subject in the form of a pharmaceutical composition. Oral dosage forms to be used are for example tablets, capsules, sachets, micropellets, granules or the like. The oral dosage forms can comprise in addition to the Mdm2i further conventional carriers or excipients used for pharmaceuticals. Examples of such carriers or excipients include, but are not limited to, disintegrants, binders, lubricants, glidants, stabilizers, and fillers, diluents, colorants, flavours and preservatives. One of ordinary skill in the art may select one or more of the aforementioned carriers with respect to the particular desired properties of the dosage form by routine experimentation and without any undue burden. The amount of each carriers used may vary within ranges conventional in the art. The following references disclose techniques and excipients used to formulate oral dosage forms. See The Handbook of Pharmaceutical Excipients, 4th edition, Rowe et al., Eds., American Pharmaceuticals Association (2003); and Remington: the Science and Practice of Pharmacy, 20th edition, Gennaro, Ed., Lippincott Williams & Wilkins (2003). The dosage forms are prepared for example by blending, granulating, compressing, compacting, filling, sieving, mixing and/or tableting.

As used herein, the term “subject” refers to an animal. Preferably, the animal is a mammal. A subject refers to for example, primates (e.g. humans), cows, sheep, goats, horses, dogs, cats, rabbits, rats, mice, fish, birds and the like. In a preferred embodiment, the subject is a human.

As used herein the term “TEAD dependent cancer” refers to any cancer in which TEAD (i.e. TEAD1, TEAD2, TEAD3 and/or TEAD4,), or a mutant or variant thereof, is known to be relevant, for example, in cancers where the Hippo pathway is genetically altered.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically or in quality of life from such treatment.

As used herein the term “reducing kidney toxicity” includes, inter alia, reduction of urine kidney injury biomarkers NGAL and/or KIM-1.

The term “a therapeutically effective amount” of a compound of the present disclosure refers to an amount of the compound of the present disclosure that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present disclosure that, when administered to a subject, is effective to (1) at least partially alleviating, inhibiting, preventing and/or ameliorating a condition, or a disorder or a disease associated with (i) hyperactivation of the YAP/TAZ-TEAD complex (ii) mediated by YAP overexpression and/or YAP amplification, or (iii) associated with YAP activity, or (iv) characterized by activity (normal or abnormal) of YAP; or (2) reducing or inhibiting the interaction of YAP and/or TAZ with TEAD. In another non-limiting embodiment, the term “a therapeutically effective amount” refers to the amount of the compound of the present disclosure that, when administered to a cell, or a tissue, or a non-cellular biological material, or a medium, is effective to at least partially reducing or inhibiting the interaction of YAP and/or TAZ with TEAD.

As used herein, the term “a,” “an,” “the” and similar terms used in the context of the present invention (especially in the context of the claims) are to be construed to cover both the singular and plural unless otherwise indicated herein or clearly contradicted by the context.

The term “comprising” encompasses “including” as well as “consisting”; e.g., a composition comprising X may consist exclusively of X or may include additional, e.g. X and Y.

In an embodiment, the TEAD inhibitor is a YAP/TAZ-TEAD protein/protein interaction inhibitor, or a pharmaceutically acceptable salt thereof. YAP/TAZ-TEAD protein/protein interaction inhibitors function by disrupting the protein-protein interaction between YAP1/WWTR1 and all four TEAD isoforms, thereby abolishing the transcriptional activity of the complex which regulates key genes involved in proliferation and survival, as explained in:
Dupont S, Morsut L, Aragona M, Enzo E, Giulitti S, Cordenonsi M, Zanconato F, Le Digabel J, Forcato M, Bicciato S, et al. (2011). Role of YAP/TAZ in mechanotransduction. Nature 474, 179-183,
Lai D, Ho K C, Hao Y, and Yang X (2011). Taxol resistance in breast cancer cells is mediated by the hippo pathway component TAZ and its downstream transcriptional targets Cyr61 and CTGF. Cancer Res. 71, 2728-2738, and
Zhao B, Ye X, Yu J, Li L, Li W, Li S, et al. (2008) TEAD mediates YAP-dependent gene induction and growth control. Genes Dev.; 22 (14): 1962-71.

In an embodiment, the TEAD inhibitor is 4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide, or a pharmaceutically acceptable salt thereof.

In an embodiment, the daily dose of the TEAD inhibitor (e.g. of 4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide) is from 15 mg to 100 mg, e.g. from 15 mg to 75 mg, e.g. 15 mg, 30 mg, 45 mg, 60 mg, 75 mg, 90 mg or 100 mg (expressed in terms of the free drug).

In an alternative embodiment, the TEAD inhibitor is

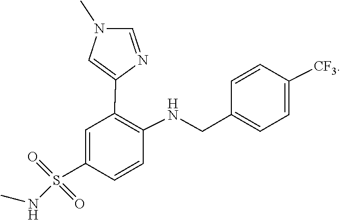 [SMILES: CNS(=O)(=O)c1ccc(NCc2ccc(C(F)(F)F)cc2)c(-c2cn(C)cn2)c1]

This inhibitor is disclosed in WO2022/159986 and WO2020/243415.

In an embodiment, the cancer is a TEAD dependent cancer, or a cancer selected from breast cancer (e.g. triple negative breast cancer), lung cancer, ovarian cancer, kidney cancer, uterine cancer, colorectal cancer, malignant pleural mesothelioma, pancreatic cancer, prostate cancer, gastric cancer, esophageal cancer, liver cancer, medulloblastoma, head and neck cancer, sarcoma, epithelioid hemangioendothelioma, ependymal tumor and bone cancer.

In an embodiment, the TEAD inhibitor is administered alongside an additional pharmaceutically active drug (combination partner). If a combination partner is present, the TEAD inhibitor is preferably provided with instructions for combined use. The compounds in the combination may be administered entirely separately. The compounds may be entirely separate pharmaceutical dosage forms. The combination partners may be pharmaceutical compositions that are sold independently of each other and where just instructions for their combined use are provided in the package equipment, e.g. leaflet or the like, or in other information e.g. provided to physicians and medical staff (e.g. oral communications, communications in writing or the like), for simultaneous or sequential use for being jointly active. The TEAD inhibitor and other pharmaceutically active drug can be provided as a fixed or a non-fixed combination of the active ingredients. The term “fixed combination” means that the active ingredients, are both administered to a patient simultaneously in the form of a single entity or dosage. In other terms: the active ingredients are present in one dosage form, e.g. in one tablet or in one capsule. The term “non-fixed combination” means that the active ingredients are both administered to a patient as separate entities either simultaneously, concurrently or sequentially with no specific time limits, wherein such administration provides therapeutically effective levels of the two compounds in the body of the patient.

Synthesis of Compound A (4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide) was originally described in PCT/IB2021/052136 (WO2021/186324), the contents of which are incorporated by reference.

### EXAMPLES

The following Examples illustrate (but are not intended to limit) the present invention:

### Example 1

Antitumor Activity, Survival and Urine Kidney Injury Markers Evaluation of Compound A in the MSTO-211H s.c. Xenograft Rat Model

Female nude rats bearing MSTO-211H subcutaneous xenografts were treated with Compound A or vehicle control for a total period of 4 weeks. All rats treated with Compound A were given a total weekly dose of 420 mg/kg Compound A p.o., albeit in four separate cohorts:
i) 60 mg/kg QD
ii) 84 mg/kg 5 days on/2 days off
iii) 120 mg/kg 1 day on/1 day off
iv) 140 mg/kg 3 days on/4 days off

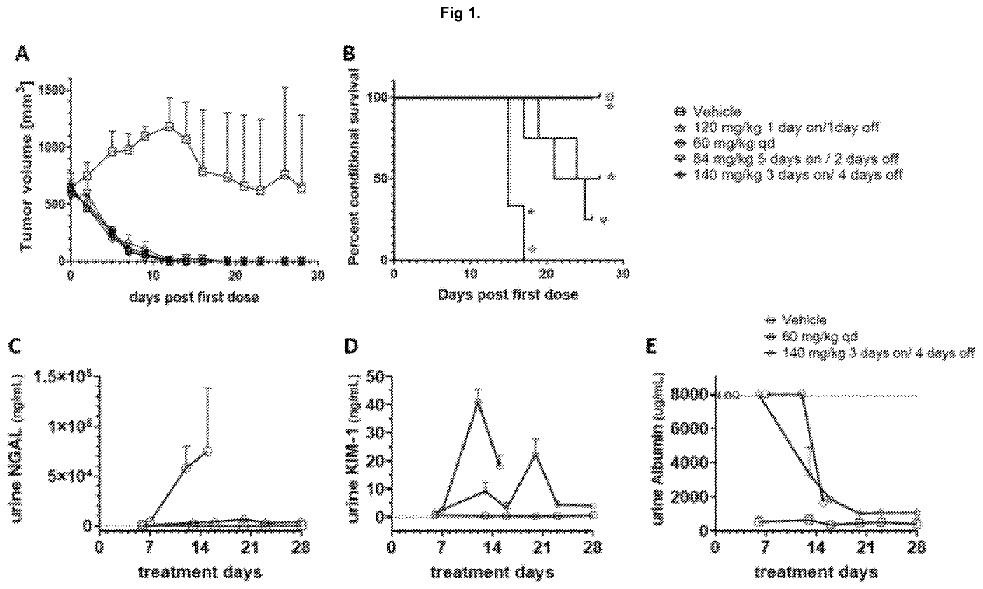

Data relating to antitumor activity (FIG. 1A), survival (FIG. 1B), and urine kidney injury marker evaluation, namely NGAL (FIG. 1C), KIM-1 (FIG. 1D) and albumin (FIG. 1E) were collected from rat samples.

A) Antitumor activity of Compound A: Some of the vehicle tumors underwent spontaneous regression when the rat immune system recovered from sub-lethal irradiation. Values are mean±SEM; sample size, (n=3-4 rats per group). The Compound A dosage regimens were all similarly effective in reducing tumor volume.

B) Animal survival *: p<0.05, significant inhibition compared to vehicle control group (Log-Rank Mantel Cox test). The toxicity of Compound A was shown to be schedule-dependent, as demonstrated by the finding that a weekly dose of 420 mg/kg/week was lethal when given in a 60 mg/kg QD schedule, but when the same weekly dose was administered with a long dosing break in a schedule of 3 days on/4 days off, all rats survived and NGAL and KIM-1 renal urine biomarker levels were lower, suggesting less kidney tubular damage.

Urine analytes. C) NGAL, D) KIM-1 and E) albumin were quantified from rat samples collected at indicated days. Sample size n=1-4 per group, mean±SEM are represented. The upper limit of quantification (LOQ) is indicated for urine albumin levels. Elevated urine kidney markers were observed for the 60 mg/kd QD cohort, which resulted in the animals being terminated after 14 to 18 days of continuous therapy. Analysis of urine quantification of NGAL, KIM-1, and albumin, revealed that renal tubular injury and protein-losing nephropathy constitute a dose limiting-toxicity in nude rats.

### Example 2

Comparative Antitumor Activity of Compound A with Daily or Intermittent Schedules in Nude Rats Bearing MSTO-211H and NCI-H226 Mesothelioma Tumors.

Efficacy studies over 3 or 4 weeks of treatment with Compound A administered p.o. with QD (plain lines) or 3 days on/4 days off intermittent schedules (dotted lines) were conducted using weekly doses of 105 or 210 mg/kg, in MSTO-211H and NCI-H226 rat models. Female nude rats bearing MSTO-211H or NCI-H226 s.c. xenografts were treated with vehicle control or Compound A. The rats treated with Compound A were split into four separate cohorts:
i) 15 mg/kg qd
ii) 35 mg/kg 3 days on/4 days off
iii) 30 mg/kg qd; and
iv) 70 mg/kg 3 days on/4 days off

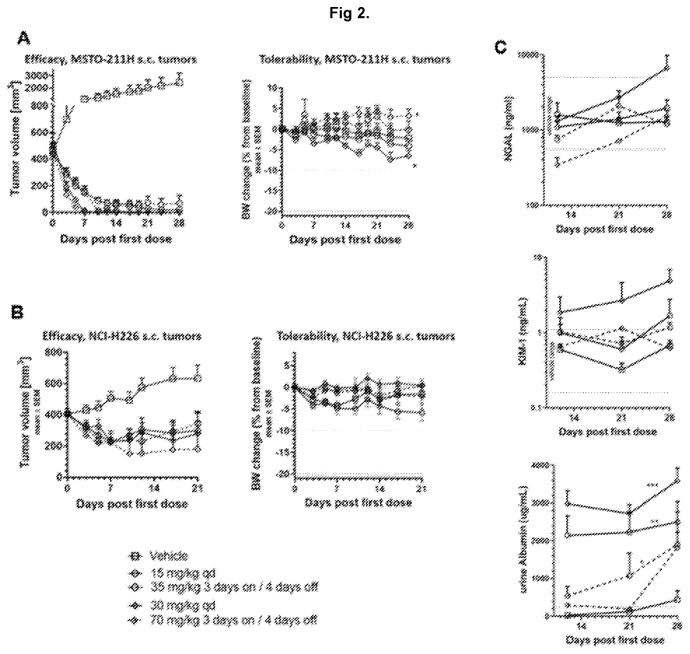

Efficacy (FIG. 2A,B, left hand side), and Tolerability (FIG. 2A, B, right hand side) data were collected. The use of intermittent dosing had no impact on anti-tumor efficacy in either MSTO-211H or NCI-H226 mesothelioma tumor models as compared to QD dosing. The total weekly dose of 210 mg/kg was non-lethal and well-tolerated with all schedules, including 30 mg/kg QD (unlike the 60 mg/kd QD dosing regimen tested in Example 1). However, as shown in FIG. 2C, the 70 mg/kg 3 days on/4 days off intermittent dosing schedule nevertheless had a reduction in albuminuria and urine kidney injury markers, NGAL and KIM-1 compared with the 30 mg/kg QD schedule to at or near baseline levels, indicating that, even at tolerated dosing levels, a 3 days on/4 days off schedule is likely to result in reduced toxicity to the kidney compared to daily dosing.

### Example 3

A study will take place characterizing the safety and tolerability of Compound A in patients with mesothelioma and other solid tumors with Hippo pathway dysregulations to assess the safety, tolerability, PK and PD of Compound A. In this study, Compound A will be administered on each of the first 3 days of a 7 day treatment cycle, and the treatment will comprise at least two treatment cycles.

1. A TEAD inhibitor or a pharmaceutically acceptable salt thereof for use in the treatment of cancer, wherein the TEAD inhibitor or pharmaceutically acceptable salt thereof is administered on each of the first 3 days of a 7 day treatment cycle, and wherein the treatment comprises at least two treatment cycles.

2. A method of treating cancer in a subject in need thereof, wherein the method comprises administering to the subject a therapeutically effective amount of a TEAD inhibitor, or a pharmaceutically acceptable salt thereof on each of the first 3 days of a 7 day treatment cycle, and wherein the treatment comprises at least two treatment cycles.

3. A method of reducing albuminuria and/or kidney toxicity in a subject undergoing treatment with a TEAD inhibitor or a pharmaceutically acceptable salt thereof, wherein the method comprises administering to the subject a therapeutically effective amount of the TEAD inhibitor, or a pharmaceutically acceptable salt thereof, on each of the first 3 days of a 7 day treatment cycle, and wherein the treatment comprises at least two treatment cycles.

4. The TEAD inhibitor or pharmaceutically acceptable salt thereof for use in the treatment of cancer according to claim 1, or the method according to claim 2 or claim 3, wherein the TEAD inhibitor is a YAP/TAZ-TEAD protein/protein interaction inhibitor or a pharmaceutically acceptable salt thereof.

5. The TEAD inhibitor or pharmaceutically acceptable salt thereof for use in the treatment of cancer according to claim 1 or claim 4, or the method according to any one of claims 2 to 4, wherein the TEAD inhibitor or salt thereof is 4-((2S,4S)-5-Chloro-6-fluoro-2-phenyl-2-((S)-pyrrolidin-2-yl)-2,3-dihydrobenzofuran-4-yl)-5-fluoro-6-(2-hydroxyethoxy)-N-methylnicotinamide or a pharmaceutically acceptable salt thereof.

6. The TEAD inhibitor or pharmaceutically acceptable salt thereof for use in the treatment of cancer according to any one of claims 1, 4 and 5, or the method according to any one of claims 2 to 5, wherein the daily dose on each administration day is from 15 mg to 500 mg.

7. The TEAD inhibitor or pharmaceutically acceptable salt thereof for use in the treatment of cancer according to claim 6, or the method according to claim 6, wherein the daily dose on each administration day is from 15 mg to 100 mg.

8. The TEAD inhibitor or pharmaceutically acceptable salt thereof for use in the treatment of cancer according to claim 6, or the method according to claim 6, wherein the daily dose on each administration day is 15, 30, 45, 60, 75 mg, 90 mg or 100 mg.

9. The TEAD inhibitor or pharmaceutically acceptable salt thereof for use in the treatment of cancer according to any one of claims 1 and 4 to 8, or the method according to any one of claims 2 to 8, wherein the cancer is a TEAD dependent cancer or is a solid tumor with NF2/LATS1/LATS2 mutations.

10. The TEAD inhibitor or pharmaceutically acceptable salt thereof for use in the treatment of cancer according to any one of claims 1 and 4 to 9, or the method according to any one of claims 2 to 9, wherein the cancer is selected from breast cancer, lung cancer, ovarian cancer, kidney cancer, uterine cancer, colorectal cancer, mesothelioma, e.g. malignant pleural mesothelioma, pancreatic cancer, prostate cancer, gastric cancer, esophageal cancer, liver cancer, medulloblastoma, head and neck cancer, sarcoma, epithelioid hemangioendothelioma, ependymal tumor and bone cancer, e.g. wherein the cancer is mesothelioma, e.g. wherein the cancer is malignant pleural mesothelioma.
